(19) United States
(12) Patent Application Publication
(10) Pub. No.: US 2021/0161917 A1
(43) Pub. Date: Jun. 3, 2021

(54) COMPOSITION FOR SUBCUTANEOUS INJECTION, CONTAINING DEOXYCHOLIC ACID, AND PREPARATION METHOD THEREFOR

(71) Applicant: INVENTAGE LAB INC., Seongnam-si (KR)

(72) Inventors: Ju Hee KIM, Seongnam-si (KR); Sang No LEE, Seoul (KR)

(21) Appl. No.: 17/048,408

(22) PCT Filed: Apr. 16, 2019
(86) PCT No.: PCT/KR2019/004587

§ 371 Date: Oct. 16, 2020

(30) Foreign Application Priority Data
Apr. 18, 2018 (KR) 10-2018-0044857
Apr. 16, 2019 (KR) 10-2019-0044378

(51) Int. Cl. A61K 31/575; A61K 9/00; A61K 9/50

(52) U.S. Cl. CPC A61K 31/575; A61K 9/5089; A61K 9/5031; A61K 9/0019

(57) ABSTRACT

The present invention relates to a composition for subcutaneous injection, containing deoxycholic acid, and a preparation method therefor. The present invention relates to a composition for non-surgical removal of localized fat deposits, the composition being micro-particles, which comprise deoxycholic acid and a biodegradable polymer, wherein the microparticles are formed such that deoxycholic acid is evenly distributed in a spherical biodegradable polymer. According to the present invention, a lipolysis effect lasts for 1 to 3 months from a single injection, a phenomenon in which surrounding tissues are destroyed during administration is prevented, and a drug release effect can be maintained at an effective amount of adipolysis concentration.

### TECHNICAL FIELD

The present disclosure relates to a composition for subcutaneous injection, containing deoxycholic acid, and a preparation method therefor. More particularly, the present disclosure relates to a composition for subcutaneous injection, comprising micro-particles containing deoxycholic acid and showing a long-lasting lipolytic effect through a single administration, and a preparation method therefor.

### BACKGROUND ART

Liposuction, also known as lipoplasty, liposculpture suction lipectomy, or simply lipo, is a cosmetic surgery that removes fat from various sites of the human body. Examples of the surgical sites include the abdomen, thighs, buttocks, neck and back of arms, and the like.

The amount of fat that can be safely removed by a single surgery is limited by several factors. Removing too much fat has a negative aspect. Abnormal lumpiness and/or dents may be observed in the skin in “over-suctioned” patients. The greater the amount of fat removed, the higher the risk of the surgery.

Injection lipolysis is a cosmetic procedure in which a drug mixture is injected into a patient for the purpose of destroying fat cells. This procedure, which generally uses phosphatidylcholine (PPC) and deoxycholate (DCA)-based drugs, stared from the use of the drug formulation as an initial intravenous injection to treat blood diseases.

PPC is phospholipids into which choline is introduced as a head group. PPC is a major component of biological membranes and can be readily obtained from a variety of readily available sources such as egg yolk or soybean, and PPC is extracted mechanically or chemically by using hexane from these sources. PPC is used to prevent fat accumulation and to treat liver failure, myocardial ischemia, cerebrovascular disease, and dementia induced by fatty liver. In addition, PPC has recently been introduced in the United States, Europe, and other countries around the world for fat dissolution for the treatment of obesity.

DCA is one of the secondary bile salts, which is a metabolic byproduct of intestinal bacteria. DCA has been used in various fields of human medicine since its discovery, and in lipolytic injection, has been widely used in combination with PPC. DCA has been used to improve the water solubility of PPC and, more recently, drugs such as amphotericin B, taxol, and diazepam. Highly purified PPC can be rapidly sterilized in combination with DCA, antimicrobial agents, benzyl alcohol, and water to form stable, mixed micelle formulations that can be used for intravenous administration.

Recently, Kythera Biopharmaceuticals, located in the United States, has been developing lipolytic injections using DCA that does not contain PPC. Based on the fact that PPC functions as an emulsifier in DCA and is used in the treatment of hyperlipidemia, PPC has been considered as an active ingredient in lipolytic injections. Among these prior art compositions, surfactants such as DCA were simply added to disperse or dissolve PPC, which is considered an active ingredient. However, it has been found that DCA is, in fact, an active ingredient in localized fat emulsification.

Since DCA is one of the strong surfactants in the bile salt family, DCA administration results in the destruction of surrounding tissues from the injection site. This is a problem caused by injecting a high concentration of injectable agent for lipolysis.

Accordingly, there is a need to develop a composition capable of maintaining a lipolytic effect for a long period of time and preventing side effects when a patient is exposed to highly concentrated drugs by an initial injectable agent.

### PRIOR ART DOCUMENTS

### Patent Documents

(Patent document 1) KR 10-2007-0110351 A1

### DISCLOSURE

### Technical Problem

An object of the present disclosure is to provide a composition for subcutaneous injection, containing deoxycholic acid and a preparation method therefor.

Another object of the present disclosure is to provide a composition for subcutaneous injection, containing deoxycholic acid which has a lipolytic effect lasting for 1 to 3 months through one injection and a preparation method therefor.

Still another object of the present disclosure is to provide a composition for subcutaneous injection, containing deoxycholic acid which prevents destruction of surrounding tissues during administration and maintains a drug-releasing effect in an effective amount of adipolysis concentration and a preparation method therefor.

### Technical Solution

In order to achieve the above object, a composition for subcutaneous injection, containing deoxycholic acid, according to an embodiment of the present disclosure is a composition for non-surgically removing localized fat deposits, wherein the composition comprises micro-particles containing deoxycholic acid and a biodegradable polymer, the micro-particles are spherical particles, have a number of pores formed therein, and the deoxycholic acid is contained in the pores.

The micro-particles may be a W₁(Water)/O (Oil)/W₂(Water) double emulsion.

The deoxycholic acid may be contained in the form of deoxycholic acid, or a pharmaceutically acceptable salt thereof.

The micro-particles may have an average diameter of 30 to 70 μm.

The micro-particles may continuously release the deoxycholic acid for 1 to 3 months to maintain a lipolytic effect.

The biodegradable polymer may be selected from the group consisting of polylactic acid, polylactide, polylactic-co-glycolic acid, polylactide-co-glycolide (PLGA), polyphosphazine, polyiminocarbonate, polyphosphoester, polyanhydride, polyorthoester, polycaprolactone, polyhydroxyvalate, polyhydroxybutyrate, polyamino acid, and a combination thereof.

The localized fat deposits may be selected from the group consisting of lower eyelid fat herniation, lipomas, lipodystrophy, fat deposits associated with cellulite, and a combination thereof.

The fat deposits are localized to a region of a subject selected from the group consisting of under eyes, under a chin, under arms, buttocks, calves, a back, thighs, ankles, a stomach, and a combination thereof.

An injectable agent for lipolysis according to another embodiment of the present disclosure comprises the composition.

A method for preparing a composition for subcutaneous injection containing deoxycholic acid according to still another embodiment of the present disclosure comprises the steps of: 1) dissolving deoxycholic acid in water to prepare a first mixture; 2) dissolving a biodegradable polymer in an organic solvent to prepare a second mixture; 3) injecting the first mixture of the step 1) into a micro-channel in a linear direction and allowing the first mixture to flow therein; 4) injecting the second mixture of the step 2) into a micro-channel formed on either side or one side so as to form a cross-point with the micro-channel in which the first mixture of the step 3) flows in the linear direction and allowing the second mixture to flow therein, and then crossing the flow of the first mixture in the linear direction with the flow of the second mixture to prepare a W₁/O emulsion in which an aqueous deoxycholic acid solution is distributed in a spherical biodegradable polymer organic solvent; 5) dissolving a surfactant in water to prepare a third mixture; 6) injecting the third mixture of the step 5) into a micro-channel formed on either side or one side so as to form a cross-point with a micro-channel in which the W₁/O emulsion of the step 4) flows in the linear direction and allowing the third mixture to flow therein, and then crossing the flow of the W₁/O emulsion in the linear direction with the flow of the third mixture to prepare a spherical W₁/O/W₂ double emulsion; 7) collecting the W₁/O/W₂ double emulsion generated at the cross-point of the step 6); 8) stirring the W₁/O/W₂ double emulsion collected in the step 7) to evaporate and remove the organic solvent present in the double emulsion; and 9) washing and drying micro-particles prepared by the W₁/O/W₂ double emulsion in the step 8), wherein the micro-particles have an average diameter of 30 to 70 μm.

The second mixture may further include a surfactant.

[symbol]

Hereinafter, the present disclosure will be described in more detail.

[symbol]

The composition for subcutaneous injection according to an embodiment of the present disclosure is a composition for non-surgically removing localized fat deposits, wherein the composition comprises micro-particles containing deoxycholic acid and a biodegradable polymer, the micro-particles are spherical particles, have a number of pores formed therein, and the deoxycholic acid is contained in the pores.

In order to solve the problem of localized fat deposits in animals by providing a non-surgical method for reducing fat deposits, the present disclosure prepares micro-particles using a biodegradable polymer, wherein a number of pores are formed in the micro-particles, and deoxycholic acid is located in the pores.

That is, the micro-particles have no pores observed in a smooth form in appearance, but have a number of pores formed therein, and the pores may be filled with deoxycholic acid, partially filled with deoxycholic acid, or contain a little deoxycholic acid attached thereto.

The present disclosure relates to the use of bile acids or a salt thereof to reduce fat in animals, preferably mammals, and more preferably humans. In an embodiment, the bile acid or a salt thereof is included in the form of deoxycholic acid or a pharmaceutically acceptable salt thereof.

The deoxycholic acid or a salt thereof refers to pharmaceutically acceptable salts of (4R)-4-((3R,5R,10 S,12 S,13R,17R)-3,12-dihydroxy-10,13 -dimethylhexadecahydro-1H-cyclic penta[a]phenanthren-17-yl)pentanoate which has an alkali metal or an ammonium ion as a cation.

The present disclosure is provided as a composition for subcutaneous injection in order to reduce the accumulation of subcutaneous fat in mammals by topically administering a pharmaceutically acceptable agent to a target site, wherein the micro-particles are contained in the composition for subcutaneous injection and the micro-particles themselves are administered locally to the target site. The micro-particles are composed of a biodegradable polymer, and have a number of pores formed therein, deoxycholic acid is distributed in the pores, and as the biodegradable polymer of the micro-particles is decomposed in subcutaneous fat, the micro-particles exerts the effect of releasing deoxycholic acid to the target site.

The present disclosure relates to a composition that uses deoxycholic acid, which is known to dissolve cellulite and fat cells associated with excess fat deposition, and can sustain a lipolytic effect for about 1 to 3 months through a single injection.

More specifically, the composition for decomposing fat according to the present disclosure is included in the form of deoxycholic acid or a pharmaceutically acceptable salt thereof, and the deoxycholic acid is not used alone, and a biodegradable polymer is used in the form of micro-particles.

That is, when prepared by using the biodegradable polymer, micro-particles contain deoxycholic acid or a pharmaceutically acceptable salt thereof therein, so that deoxycholic acid or pharmaceutically acceptable salt thereof is characterized by being distributed in the pores in the prepared micro-particles.

The biodegradable polymer is a polymer that can be decomposed in the body, and the decomposed polymer is characterized by not being harmful to the human body. Accordingly, the micro-particles are slowly decomposed in the body, and at this time, may exert an effect of releasing deoxycholic acid or a pharmaceutically acceptable salt thereof contained in the exposed pores.

In order to continuously release this deoxycholic acid or a pharmaceutically acceptable salt thereof for 1 to 3 months, the micro-particles have an average diameter of 30 to 70 μm. If the micro-particles have the average diameter of less than 30 μm, the sizes of the particles are too small, and the micro-particles are highly likely to being ingested by macrophages after injection into the body increases, thereby affecting the release of effective drugs and absorption in vivo.

In addition, if the micro-particles have the average diameter of more than 70 μm, foreign body sensation and pain may be increased when the injectable agent is administered, and the particle size distribution of the prepared particles may be increased, thereby making it difficult to prepare micro-particles having a uniform particle size.

Preferably, the micro-particles of the present disclosure are prepared according to the preparation method described later, and in this case, a micro-channel is used. As one condition for controlling the sizes of the particles, a width (w) and a depth (d) of the cross section of the micro-channel need to be adjusted within the range of an average diameter of the micro-particles to be prepared, and a certain length ratio.

More specifically, since the width (w) of the cross section of the channel is in the range of 0.7 to 1.3 with respect to an average diameter (d′) of the micro-particles, the width of the cross section of the channel needs be set to 70 to 130 μm to prepare the micro-particles with 100 μm of the average diameter (d′).

In addition, since the depth (d) of the cross section of the channel is in the range of 0.7 to 1.3 with respect to the average diameter (d′) of the micro-particles, the height of the cross section of the channel need to be set to 70 to 130 μm to prepare the micro-particles with 100 μm of the average diameter (d′).

In order to prepare the micro-particles of the present disclosure, a liquid mixture needs to be injected into the micro-channel, and at this time, it is characterized in that micro-particles each having a desired size may be prepared by adjusting the width (w) and depth (d) of the cross section of the micro-channel as described above.

The biodegradable polymer is selected from the group consisting of polylactic acid, polylactide, polylactic-co-glycolic acid, polylactide-co-glycolide (PLGA), polyphosphazine, polyiminocarbonate, polyphosphoester, polyanhydride, polyorthoester, polycaprolactone, polyhydroxyvalate, polyhydroxybutyrate, polyamino acid, and a combination thereof and is not limited to those as indicated above.

The localized fat deposits may be selected from the group consisting of lower eyelid fat herniation, lipomas, lipodystrophy, fat deposits associated with cellulite, and a combination thereof.

The fat deposits may be localized to a region of a subject selected from the group consisting of under eyes, under a chin, under arms, buttocks, calves, a back, thighs, ankles, a stomach, and a combination thereof. The subject is preferably under chin, and more specifically, the present disclosure is to eliminate localized fat deposits that cause a double chin.

The injectable agent for lipolysis according to another embodiment of the present disclosure may comprise the composition for subcutaneous injection.

The lipolytic composition is directly injected into the subcutaneous fat layer of the user's body, selectively destroying fat cells, causing a reduction in fat volume, and reducing the sizes of the face and body shape and correcting the face and body shape. At this time, the lipolytic composition may be injected into a subcutaneous fat layer such as a user's double chin site.

That is, when the lipolytic composition is injected into human tissue, the micro-particles are gradually decomposed, and release deoxycholic acid or its salt, and at this time, the cells of the tissue injected with deoxycholic acid or a salt thereof (for example, fat cells and the like) are swelled by a difference in osmotic pressure, and the cells swelled by a difference in osmotic pressure make the destruction of fat cells by the deoxycholic acid more effective.

In this way, by directly injecting the lipolytic composition into the user's double chin site with relatively few motor nerves, it is possible to maximize the lipolytic effect and reduce side effects due to permanent damage to the motor nerves.

Due to the action of the lipolytic composition of the present disclosure injected into the subcutaneous fat layer, the fat cell membrane is destroyed and the fat is changed to be water-soluble. Thereafter, the fat changed to water-soluble may be discharged to the outside of the body through urine or sweat.

A method for preparing a composition for decomposing the fats according to still another embodiment of the present disclosure comprises the steps of: 1) dissolving deoxycholic acid in water to prepare a first mixture; 2) dissolving a biodegradable polymer in an organic solvent to prepare a second mixture; 3) injecting the first mixture of the step 1) into a micro-channel in a linear direction and allowing the first mixture to flow therein; 4) injecting the second mixture of the step 2) into a micro-channel formed on either side or one side so as to form a cross-point with the micro-channel in which the first mixture of the step 3) flows in the linear direction and allowing the second mixture to flow therein, and then crossing the flow of the first mixture in the linear direction with the flow of the second mixture to prepare a W₁/O emulsion in which an aqueous deoxycholic acid solution is distributed in a spherical biodegradable polymer organic solvent; 5) dissolving a surfactant in water to prepare a third mixture; 6) injecting the third mixture of the step 5) into a micro-channel formed on either side or one side so as to form a cross-point with the micro-channel in which the W₁/O emulsion of the step 4) flows in the linear direction and allowing the third mixture to flow therein, and then crossing the flow of the W₁/O emulsion in the linear direction with the flow of the third mixture to prepare a spherical W₁/O/W₂ double emulsion; 7) collecting the W₁/O/W₂ double emulsion generated at the cross-point of the step 6); 8) stirring the W₁/O/W₂ double emulsion collected in the step 7) to evaporate and remove the organic solvent present in the double emulsion; and 9) washing and drying micro-particles prepared by the W₁/O/W₂ double emulsion in the step 8).

A mixture containing deoxycholic acid may be prepared by dissolving the deoxycholic acid in water, a mixture containing a biodegradable polymer may be prepared by dissolving the biodegradable polymer in an organic solvent, each of the resulting mixtures may be injected into a micro-channel and allowed to flow therein, and a W₁/O emulsion may be prepared at a cross-point at which the flow of the mixture containing deoxycholic acid and the flow of the mixture containing the biodegradable polymer cross. Thereafter, a spherical W₁/O/W₂ double emulsion may be prepared by injecting a mixture obtained by dissolving a surfactant in water so as to cross with the flow the W₁/O emulsion in the linear direction, and then crossing the flow of the W₁/O emulsion with the flow of the mixture containing the surfactant.

More specifically, when injected into a micro-channel in a linear direction, the first mixture is injected under a constant pressure condition and allowed to flow at a constant flow rate, and at this time, the pressure condition is 100 to 500 mbar, preferably 300 mbar, but is not limited thereto. In addition, when injected into a micro-channel in either side or one side, the second mixture is injected under a constant pressure condition and allowed to flow at a constant flow rate, and at this time, the pressure condition is 500 to 1000 mbar, preferably 700 mbar, but is not limited thereto.

In addition, when injected into a micro-channel in either side or one side, the third mixture is injected under a constant pressure condition and allowed to flow at a constant flow rate, and at this time, the pressure condition is 1,000 to 2,000 mbar, preferably 1,500 mbar, but is not limited thereto.

That is, in order to make the flow of the second mixture forming a cross-point with the flow of the first mixture flow at a faster flow rate than the first mixture injected into the micro-channel in the linear direction, the second mixture is allowed to flow under higher pressure conditions.

Likewise, in order to make the flow of the third mixture forming a cross-point with a micro-channel in which the W₁/O emulsion flows in the linear direction at a faster flow rate, the third mixture is allowed to flow under higher pressure conditions.

As described above, by varying the flow rate of the first mixture and the second mixture, and making the flow rate of the third mixture faster than that of the mixture containing the W₁/O emulsion, the second mixture having a relatively faster flow rate compresses the first mixture at the point where the flow of the first mixture meets the flow of the second mixture, and at this time, the W₁/O emulsion is formed due to the repulsive force of the first mixture and the second mixture.

The W₁/O emulsion is in a state in which deoxycholic acid contained in the first mixture is dissolved in a solvent by the flow of the second mixture faster than the flow of the first mixture, that is, the first mixture itself is compressed into a spherical shape by the flow of the second mixture.

When the W₁/O emulsion is formed in this way, the formed W₁/O emulsion is contained in the second mixture and flows along the micro-channel in the linear direction.

Thereafter, when the W₁/O emulsion flows in the linear direction, the flow of the third mixture, which is faster than the flow rate of the flow of the W₁/O emulsion, is crossed to form a W₁/O/W₂ double emulsion.

The W₁/O/W₂ double emulsion is in an oil phase, that is, a biodegradable polymer uniformly dissolved in an organic solvent has a form in which a number of deoxycholic acids dissolved in an aqueous phase are encapsulated in a spherical shape, and is again transformed into a spherical shape by the flow of the third mixture to form a W₁/O/W₂ double emulsion. More specifically, the W₁/O/W₂ double emulsion has a form in which a number of deoxycholic acid solutions are, in a spherical shape, encapsulated in a spherical biodegradable polymer capsule.

Thereafter, in the step of collecting the micro-particles, the micro-particles are collected in a water bath containing the third mixture, thereby preventing aggregation between the initially generated micro-particles.

The step of collecting the micro-particles is to use the third mixture, that is, a mixed solution of surfactant and water, and after preparing the third mixture, a part thereof is injected into the micro-channel, and the other part thereof is moved to the water bath for collecting the micro-particles, and is used to prevent aggregation between the collected micro-particles.

After the step of collecting the micro-particles, in the step of stirring the micro-particles collected in the water bath, the micro-particles are stirred at a constant temperature condition and a stirring speed to evaporate and remove the organic solvent present on the surfaces of the micro-particles.

At this time, the step of stirring the micro-particles is performed in order of a first stirring step under the stirring conditions of a speed of 300 to 500 rpm for 1 to 2 hours at 16 to 18° C.; after the first stirring step, a second stirring step under the stirring conditions of a speed of 500 to 700 rpm for 2 to 4 hours at 30 to 50° C.; and after the second stirring step, at third stirring step under the stirring conditions of a speed of 500 to 700 rpm for 1 to 2 hours at 0 to 15° C.

In the case of the first and second stirring steps, after the micro-particles are stirred while the temperature is increased, the evaporation rate of the solvent present in the surfaces and inner pores of the micro-particles may be adjusted. That is, by gradually evaporating the solvent present in the micro-particles, micro-particles each having a smooth surface and containing only deoxycholic acid in the internal pores may be prepared.

In addition, as the stirring process is performed at a low temperature in the final third step, the surfaced of the micro-particles may be evenly maintained after stirring, and the micro-particles may maintain the properties thereof evenly after drying.

That is, before the stirring process, the inside each of the micro-particles is in a state in which a number of deoxycholic acid solutions are encapsulated in a spherical shape, and it is important to remove an internal solution and leave only deoxycholic acid.

At this time, when the stirring processes are performed by varying the temperature conditions as in the first to the third stirring steps, an external solvent is removed, so that the surfaces of the micro-particles become smooth and only the internal solvent is removed to form a number of pores in the micro-particles, and only deoxycholic acid remains in the pores.

More specifically, preferably, the first stirring step is performed at 17° C. for 1 hour, and the second stirring step is performed at 40° C. for 3 hours. Thereafter, the third stirring step is performed at 10° C. for 1 hour.

The temperature when the first mixture to the third mixture flows through the micro-channels is 16 to 18° C., preferably 17° C. That is, after flowing through the micro-channel and forming the cross-point to generate micro-particles, the first mixture to the third mixture are kept at a constant low temperature of 16 to 18° C. until the collected micro-particles are subject to the first stirring step. It is possible to prepare and maintain spherical particles only by maintaining a low temperature during the preparing process of micro-particles. That is, in the case of non-low temperature conditions, it is difficult to prepare particles each having a certain spherical shape.

Finally, in the step of washing and drying the micro-particles, the micro-particles from which all the organic solvents on the surfaces thereof are removed by stirring are washed several times with purified water filtered through sterilization to remove the surfactant remaining on the micro-particles, and then freeze-dried.

The micro-particles may be prepared by injecting mixtures into micro-channel formed on a wafer and allowing the mixtures to flow therein.

More specifically, aluminum is deposited on a silicon wafer using an e-beam evaporator, and a photoresist is patterned on the aluminum using a photolithography technique. Thereafter, aluminum is etched using the photoresist as a mask, the photoresist is removed, silicon is etched with deep ion reactive etching (DRIE) using aluminum as a mask, and then, glass is anodic bonded to the wafer to be sealed after removing the aluminum, thereby preparing the micro-channel.

The organic solvent of the present disclosure is one that is not mixed with water, for example, one or more selected from the group consisting of chloroform, chloroethane, dichloroethane, trichloroethane, and a mixture thereof, preferably dichloromethane, but is not limited thereto, and is an organic solvent capable of dissolving a biodegradable polymer, and is not limited thereto, and any organic solvent that can be easily selected by one skilled in the art can be used.

As the surfactant of the present disclosure, any surfactant may be used without limitation as long as the surfactant can help biodegradable polymer solution form a stable emulsion. Specifically, the surfactant may be one or more selected from the group consisting of nonionic surfactants, anionic surfactants, cationic surfactants, and a mixture thereof, and more specifically, one or more selected from the group consisting of methylcellulose, polyvinylpyrrolidone, lecithin, gelatin, polyvinyl alcohol, polyoxyethylene sorbitan fatty acid ester, polyoxyethylene castor oil derivative, sodium lauryl sulfate, sodium stearate, ester amine, linear diamine, fatty amine, and a mixture thereof, preferably polyvinyl alcohol, and is not limited thereto.

The second mixture may further contain a surfactant. In general, the surfactant is dissolved in water and used, but in order to prepare the W₁/O/W₂ double emulsion, the surfactant is additionally dissolved in the second mixture, and enables the preparation of the W₁/O/W₂ double emulsion having a uniform shape only during preparation.

Preferably, the second mixture may be prepared by dissolving a biodegradable polymer and a second surfactant in an organic solvent, and may contain 10 to 20% by weight of the biodegradable polymer, 1 to 3% by weight of the surfactant, and the remaining organic solvent.

When used within the above range, the surfactant enables the preparation of the W₁/O/W₂ double emulsion having a uniform shape, and enables the production of the W₁/O/W₂ double emulsion as a long-lasting formulation.

“Patient”, “subject”, “individual”, and the like in the present disclosure are used interchangeably in the present specification, and refer to any animal or cell thereof, which may be adapted to a method described in the present specification, regardless of in vitro or in situ. In certain non-limiting embodiments, the patient, subject or individual is a human.

“Composition” or “pharmaceutical composition” in the present disclosure refers to a mixture of at least one compound of the present disclosure and other chemical components such as carriers, stabilizers, diluents, dispersants, suspending agents, thickening agents, and/or excipients. The pharmaceutical composition facilitates administration of the compound to an organism.

“Effective amount”, “pharmaceutically effective amount” and “therapeutically effective amount” in the present disclosure refer to an amount which is non-toxic but sufficient to provide a desired biological result. The result may be a reduction and/or alleviation of signs, symptoms, or causes of disease, or any other desired alteration of the biological system. The appropriate therapeutic amount in any individual case may be determined by one skilled in the art using routine experimentation.

“Local administration” in the present disclosure refers to the administration of a pharmaceutical ingredient to or around a muscle or subdermal location of a patient by a non-systemic route. Thus, topical administration excludes administration through systemic routes such as intravenous or oral administration.

“Pharmaceutically acceptable” in the present disclosure refers to properties and/or substances allowable to a preparing pharmaceutical chemist from the pharmacological/toxicological point of view and from the physical/chemical point of view with respect to the composition, formulation, stability, patient acceptability, and bioavailability. “Pharmaceutically acceptable carrier” refers to a medium that is administered without interfering with the effectiveness of the biological activity of active ingredient(s) and is not toxic to a host.

The “composition for subcutaneous injection, containing deoxycholic acid” of the present disclosure comprises micro-particles wherein the micro-particles are spherical particles, each have a smooth appearance and have a number of pores formed therein, and the deoxycholic acid is contained in the pores, and the micro-particles are mixed with water for injection, and the micro-particles themselves may be administered subcutaneously.

### Advantageous Effects

According to the present disclosure, it is possible to provide a composition for subcutaneous injection, containing deoxycholic acid, which maintains the lipolytic effect for 1 to 3 months through one injection, prevents destruction of surrounding tissues when administered, and sustains the drug-releasing effect in an effective amount of adipolysis concentration, and a preparation method therefor.

### BRIEF DESCRIPTION OF DRAWINGS

FIG. 1 is a microscope photograph of a W₁/O/W₂ double emulsion according to an embodiment of the present disclosure.

FIG. 2 is a microscope photograph of the W₁/O/W₂ double emulsion according to an embodiment of the present disclosure.

FIG. 3 is an SEM photograph of micro-particles according to pressure conditions during injection into a micro-channel according to an embodiment of the present disclosure.

FIG. 4 is an SEM photograph of micro-particles when a surfactant is contained in a second mixture according to an embodiment of the present disclosure.

FIG. 5 is an SEM photograph of micro-particles when a surfactant is not contained in the second mixture according to an embodiment of the present disclosure.

FIG. 6 is an SEM photograph of micro-particles depending on stirring conditions according to an embodiment of the present disclosure.

FIG. 7 is an SEM photograph of micro-particles depending on stirring conditions according to an embodiment of the present disclosure.

FIG. 8 is an SEM photograph of micro-particles depending on stirring conditions according to an embodiment of the present disclosure.

FIG. 9 is a result of the drug release time of the micro-particles according to an embodiment of the present disclosure.

### BEST MODE

The present disclosure is a composition for non-surgically removing localized fat deposits, and relates to a composition for subcutaneous injection, containing deoxycholic acid, wherein the composition comprises micro-particles containing deoxycholic acid and a biodegradable polymer, the micro-particles are spherical particles, have a smooth appearance, each has a number of pores formed therein, and the deoxycholic acid is contained the pores.

### Mode For Invention

Hereinafter, embodiments of the present disclosure will be described in detail so that those skilled in the art can easily carry out the present disclosure. However, the present disclosure may be implemented in various different forms and is not limited to the embodiments described herein.

### Preparation Example 1

Preparation of Micro-Particles Containing Deoxycholic Acid

0.5 g of deoxycholic acid was dissolved in 20 ml of water to prepare a first mixture. Polylactide-co-glycolide (PLGA) and Span 80 were dissolved in dichloromethane to prepare a second mixture. The second mixture was comprised of 15% by weight of PLGA, 2% by weight of Span 80 and the remaining dichloromethane.

Polyvinyl alcohol, which is a surfactant, was mixed with water to prepare a third mixture containing 0.25% by weight of polyvinyl alcohol.

The first mixture, the second mixture, and the third mixture were each injected into micro-channel formed on a silicon wafer and allowed to flow therein. The first mixture was injected at a pressure of 300 mbar and allowed to flow in a linear direction, and the second mixture was injected at a pressure of 700 mbar from the side so as to form a cross-point with the flow of the first mixture and allowed to flow therein.

The flow of the first mixture and the flow of the second mixture were crossed to prepare a W₁/O emulsion. The W₁/O emulsion was allowed to flow again in a linear direction, and the flow of the third mixture was injected from the side at a pressure of 1,500 mbar so as to cross the flow of the W₁/O emulsion with the flow of the third mixture and allowed to flow therein

A W₁/O/W₂ emulsion generated at the cross-point where the flow of the W₁/O emulsion formed by crossing the flow of the first mixture with the flow of the second mixture meets, the flow of the third mixture was collected in a water baht containing the third mixture.

The W₁/O/W₂ emulsion collected in the water bath was firstly stirred at 17° C. for 1 hour at a speed of 400 rpm, and then secondly stirred for 3 hours at a speed of 600 rpm with the temperature raised to 40° C., and was thirdly stirred at a speed of 600 rpm for 1 hour with the temperature lowered to 10° C.

After the stirring was completed, micro-particles were washed several times with purified water filtered through sterilization, and freeze-dried to prepare final micro-particles.

### Preparation Example 2

Preparation Example 2 was performed in the same manner as in Preparation Example 1 except that no span 80 was added when the second mixture is prepared.

### Preparation Example 3 to Preparation Example 7

The first mixture, the second mixture, and the third mixture were injected, by adjusting the pressure conditions as shown in the following table.

TABLE 1

| | Preparation | Preparation | Preparation | Preparation | Preparation |
|---|---|---|---|---|---|
| | Example 3 | Example 4 | Example 5 | Example 6 | Example 7 |
| The first | 100 | 500 | 50 | 300 | 300 |
| mixture | | | | | |
| The second | 500 | 900 | 700 | 400 | 700 |
| mixture | | | | | |
| The third | 1300 | 1700 | 1500 | 1500 | 1200 |
| mixture | | | | | |

(Unit: mbar)

### Preparation Example 8

Preparation Example 8 was performed in the same manner as in Preparation Example 1 except that the temperatures for the first, second and third stirring processes were each maintained at 17° C. upon the stirring process.

### Preparation Example 9

Preparation Example 9 was performed in the same manner as in Preparation Example 1 except that the temperatures for the first, second and third stirring processes were each maintained at 40° C. upon the stirring process.

### Preparation Example 10

Preparation Example 10 was performed in the same manner as in Preparation Example 1 except that the temperatures for the first, second and third stirring processes were each maintained at 10° C. upon the stirring process.

### Experimental Example 1: Whether or Not Uniform Micro-Particles Are Generated

For the micro-particles prepared as in each of Preparation Example 1 and Preparation Examples 3 to 7, the stirring process was performed, and before freeze-drying, the inner shape of the W₁/O/W₂ double emulsion was observed through a microscope photograph.

The results observed with the naked eye through the microscope photograph are as shown in Table 2 below, and the results are classified according to the following criteria.

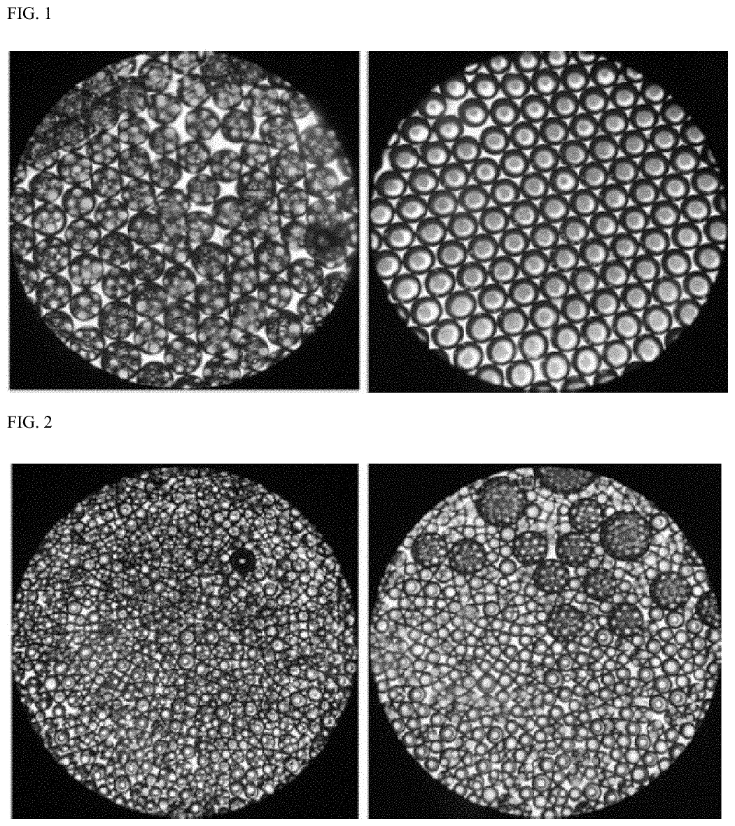

The case where the first mixture (W₁ phase) is uniformly contained in an oil phase, as shown in FIG. 1, was indicated by O, and the case where the first mixture (W₁ phase) is non-uniformly contained as shown in FIG. 2 was indicated by X.

TABLE 2

| | Preparation | Preparation | Preparation | Preparation | Preparation | Preparation |
|---|---|---|---|---|---|---|
| | Example 1 | Example 3 | Example 4 | Example 5 | Example 6 | Example 7 |
| Uniform | ◯ | ◯ | ◯ | X | X | X |
| micro-particles | | | | | | |
| or not | | | | | | |

As shown in Table 2 above, it will be said that when the first mixture, the second mixture, and the third mixture are injected into the micro-channel, there is a big difference as to whether the first mixture is uniformly encapsulated in the oil phase, depending on the pressure condition.

That is, upon injection within the range of the pressure condition of the present disclosure, deoxycholic acid may be uniformly contained in the micro-particles, but upon injection out of the range of the pressure condition of the present disclosure, deoxycholic acid may not be uniformly contained in the micro-particles, and the drug release period thus becomes impossible to control.

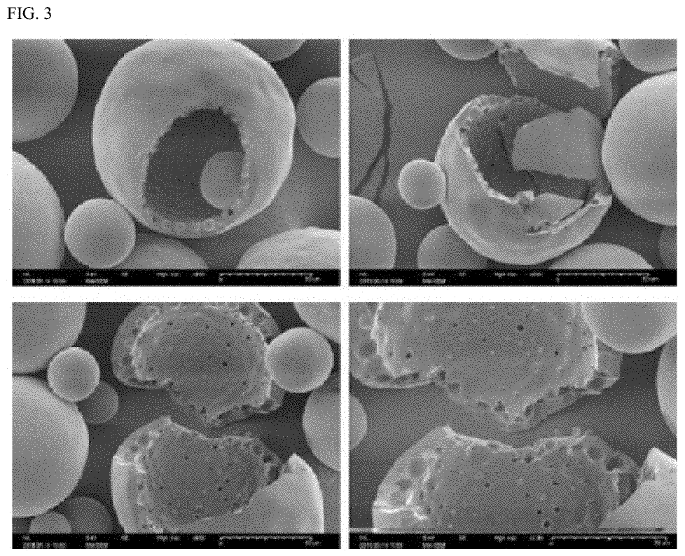

The result of taking an SEM photograph of the inner shapes of the micro-particles formed unevenly as shown in FIG. 2 is shown in FIG. 3. Referring to FIG. 3, it can be seen that the internal pores are very non-uniformly formed.

The internal pores formed non-uniformly means that the amounts of deoxycholic acid present in the pores are very small or very large, and thus it is impossible to control the drug release effect within a desired period.

[symbol]

### Experimental Example 2: Whether or Not Micro-Particles Are Generated

After the micro-particles were prepared as in Preparation Example 1 and Preparation Example 2, the properties of the prepared micro-particles were measured by SEM photographs, and the results thereof were confirmed.

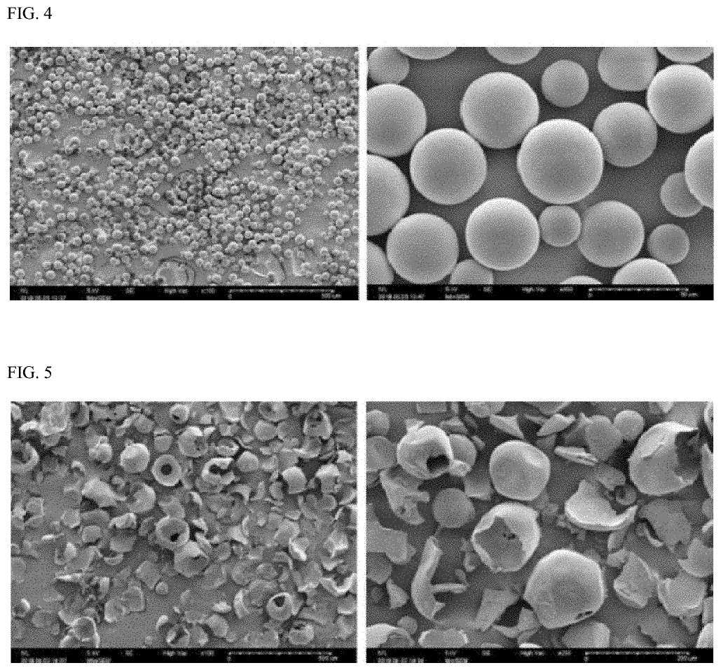

The results are shown in FIGS. 4 and 5.

It was confirmed that when the surfactant was contained upon the preparation of the second mixture as shown in FIG. 4, micro-particles having even particles were generated, but in the case of Preparation Example 2, the micro-particles were broken, and thus uniform spherical micro-particles was impossible to prepare, as shown in FIG. 5.

If a surfactant is contained in the second mixture, when the W₁/O emulsion is formed, the first mixture in the aqueous phase is encapsulated in a spherical shape in the oil phase, wherein the surfactant at the interface plays a role in helping the first mixture be maintained in a spherical shape.

Thus, as in Preparation Example 2, if the surfactant was not contained in the second mixture, when the W₁/O emulsion was formed, the first mixture was not smoothly maintained in the spherical shape, and thereafter the W₁/O/W₂ double emulsion was formed, and cracking occurred during stirring and freeze-drying, making it impossible to prepare micro-particles in the spherical shape.

On the other hand, when the W₁/O emulsion of Preparation Example 1 was formed, the surfactant present at the interface was separated from the inside to the outside in the stirring process and freeze-drying process, and thus did not exist in the finally prepared micro-particles.

[symbol]

### Experimental Example 3: Difference Between Micro-Particles Depending on Stirring Conditions

In order to confirm the changes in the properties of the micro-particles depending on the stirring conditions, SEM photographs of the micro-particles prepared in the same manner as in Preparation Example 1 and Preparation Examples 8 to 10 were confirmed.

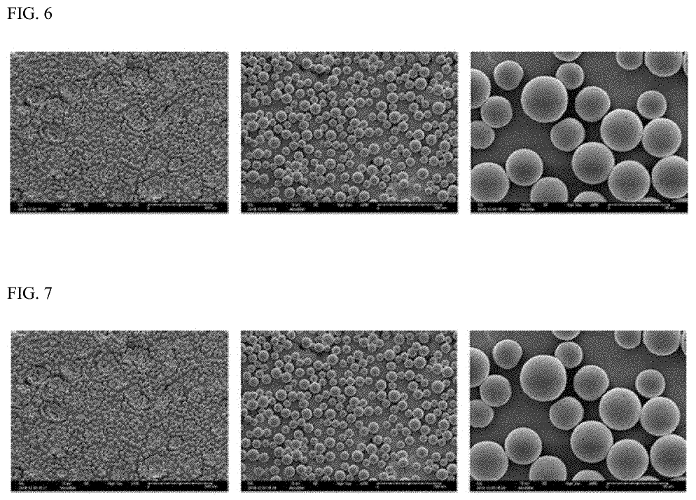

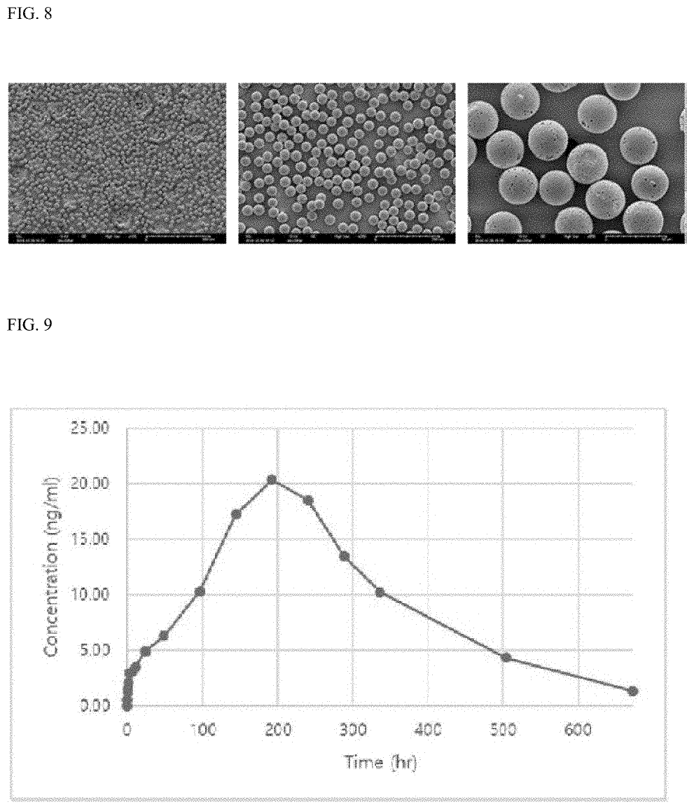

The experimental results are shown in Table 3 and FIGS. 6 to 8.

TABLE 3

| | Preparation | Preparation | Preparation | Preparation |
|---|---|---|---|---|
| | Example 1 | Example 8 | Example 9 | Example 10 |
| Uniform micro- | ◯ | X | X | X |
| particles or not | | | | |

It was confirmed that in the case of Preparation Example 1, as shown in FIG. 6, not only micro-particles each having an even particle diameter were prepared, but also micro-particles that each have an even surface and do not cause aggregation between particles were prepared. It was confirmed that in the case of Preparation Examples 8 to 10, unlike Preparation Example 1, there is a problem in that aggregation between particles occurs or pores are generated on the surfaces of the particles so that the surface thereof are not formed evenly.

That is, the surfaces thereof may not be formed evenly and the aggregation phenomenon between particles may occur depending on the stirring conditions. Thus, only when the stirring process is performed within the range of the stirring conditions of the present disclosure, it is possible to prepare micro-particles with even surfaces and no aggregation therebetween.

[symbol]

### Experimental Example 4: Experiment on Concentration of Micro-Particles in Blood

The micro-particles of Preparation Example 1 were mixed with water for injection to prepare a long-lasting injection formulation, administered to a beagle, and a blood sample thereof was collected to measure the concentration of deoxycholic acid in blood over time.

The drug release test results are shown in FIG. 9 below.

As a result of the experiment, it can be confirmed that since the micro-particles of Preparation Example 1 not only exhibited an effective concentration in blood, even at an initial stage, but also exhibited the effective concentration level in blood even after 600 hours, the micro-particles had an effect of releasing deoxycholic acid for a long time.

Based on the above experimental results, it will be said that the micro-particles according to an embodiment of the present disclosure may exhibit the sustained release effect of deoxycholic acid for 1 to 3 months.

[symbol]

Although the preferred embodiments of the present disclosure have been described in detail above, the scope of the present disclosure is not limited thereto, and various modifications and improvements made by those skilled in the art using the basic concept of the present disclosure defined in the following claims also belong to the scope of rights of the present disclosure.

### INDUSTRIAL APPLICABILITY

The present disclosure relates to a composition for subcutaneous injection, containing deoxycholic acid, and a preparation method therefor. More particularly, the present disclosure relates to a composition for subcutaneous injection, comprising micro-particles containing deoxycholic acid and showing a long-lasting lipolytic effect through a single administration, and a preparation method therefor.

1. A composition for subcutaneous injection, containing deoxycholic acid, the composition being for non-surgically removing localized fat deposits,
wherein the composition comprises micro-particles containing deoxycholic acid and a biodegradable polymer,
the micro-particles are spherical particles, each have a smooth appearance, and have a number of pores formed therein, and
the deoxycholic acid is contained in the pores.

2. The composition of claim 1, wherein the micro-particles are a W₁/O/W₂ double emulsion.

3. The composition of claim 1, wherein the deoxycholic acid is contained in the form of deoxycholic acid, or a pharmaceutically acceptable salt thereof.

4. The composition of claim 1, wherein the micro-particles have an average diameter of 30 to 70 μm.

5. The composition of claim 1, wherein the micro-particles continuously release the deoxycholic acid for 1 to 3 months to maintain a lipolytic effect.

6. The composition of claim 1, wherein the biodegradable polymer is selected from the group consisting of polylactic acid, polylactide, polylactic-co-glycolic acid, polylactide-co-glycolide (PLGA), polyphosphazine, polyiminocarbonate, polyphosphoester, polyanhydride, polyorthoester, polycaprolactone, polyhydroxyvalate, polyhydroxybutyrate, polyamino acid, and a combination thereof.

7. The composition of claim 1, wherein the localized fat deposits are selected from the group consisting of lower eyelid fat herniation, lipomas, lipodystrophy, fat deposits associated with cellulite, and a combination thereof.

8. The composition of claim 1, wherein the fat deposits are localized to a region of a subject, selected from the group consisting of under eyes, under a chin, under arms, buttocks, calves, a back, thighs, ankles, a stomach, and a combination thereof.

9. An injectable agent for lipolysis, comprising the composition of claim 1.

10. A method for preparing a composition for subcutaneous injection, containing deoxycholic acid, the method comprising the steps of:
1) dissolving deoxycholic acid in water to prepare a first mixture;
2) dissolving a biodegradable polymer in an organic solvent to prepare a second mixture;
3) injecting the first mixture of the step 1) into a micro-channel in a linear direction and allowing the first mixture to flow therein;
4) injecting the second mixture of the step 2) into a micro-channel formed on either side or one side so as to form a cross-point with a micro-channel in which the first mixture of the step 3) flows in the linear direction and allowing the second mixture to flow therein, and then crossing the flow of the first mixture in the linear direction with the flow of the second mixture to prepare a W₁/O emulsion in which an aqueous deoxycholic acid solution is distributed in a spherical biodegradable polymer organic solvent;
5) dissolving a surfactant in water to prepare a third mixture;
6) injecting the third mixture of the step 5) into a micro-channel formed on either side or one side so as to form a cross-point with a micro-channel in which the W₁/O emulsion of the step 4) flows in the linear direction and allowing the third mixture to flow therein, and then crossing the flow of the W₁/O emulsion in the linear direction with the flow of the third mixture to prepare a spherical W₁/O/W₂ double emulsion;
7) collecting the W₁/O/W₂ double emulsion generated at the cross-point of the step 6);
8) stirring the W₁/O/W₂ double emulsion collected in the step 7) to evaporate and remove the organic solvent present in the double emulsion; and
9) washing and drying micro-particles prepared by the W₁/O/W₂ double emulsion in the step 8),
wherein the micro-particles have an average diameter of 30 to 70 μm.

11. The method of claim 10, wherein the second mixture further contains a surfactant.
